(19) United States
(12) Patent Application Publication
(10) Pub. No.: US 2016/0310589 A1
(43) Pub. Date: Oct. 27, 2016

(54) Glucopyranosyl Lipid A and Allergen Formulations For Sublingual Administration

(71) Applicant: Sanofi, Paris (FR)

(72) Inventors: Jerome Arigon, Paris (FR); Pierre Calichiama, Paris (FR); Pierre DeMontigny, Bridgewater, NJ (US); El-Bdaoui Haddad, Bridgewater, NJ (US); Yan He, Bridgewater, NJ (US); Chris Ho, Bridgewater, NJ (US)

(21) Appl. No.: 15/136,440

(22) Filed: Apr. 22, 2016

Related U.S. Application Data
(60) Provisional application No. 62/151,922, filed on Apr. 23, 2015.
(60) Provisional application No. 62/194,067, filed on Jul. 17, 2015.
(60) Provisional application No. 62/303,224, filed on Mar. 3, 2016.

(30) Foreign Application Priority Data
Mar. 24, 2016 (EP) 16305344.0

(51) Int. Cl. A61K 39/35; A61K 9/00; A61K 39/39

(52) U.S. Cl. CPC A61K 39/35; A61K 39/39; A61K 9/006; A61K 2039/55572

(57) ABSTRACT

The present application relates to compositions and methods for treating allergy, e.g., peanut allergy, in patients. The methods involve administering glucopyranosyl lipid adjuvant (GLA) and peanut allergen(s) to the patient via a sublingual route.

### CROSS-REFERENCE TO RELATED APPLICATIONS

This application claims priority to U.S. Provisional Application No. 62/151,922, filed Apr. 23, 2015, U.S. Provisional Application No. 62/194,067, filed Jul. 17, 2015, and U.S. Provisional Application No. 62/303,224, filed Mar. 3, 2016, each of which is incorporated herein by reference in its entirety.

### TECHNICAL FIELD

This invention relates to treatments for allergies, e.g., peanut allergies, and to useful compositions comprising Glucopyranosyl Lipid A (GLA) and allergens, such as peanut allergens, for allergy treatments.

### BACKGROUND

Peanut allergy is characterized by an aberrant T-helper-type 2 (Th-2) immune response towards the normally innocuous antigens present in peanuts. The prevalence of peanut allergy in North American school-aged children is approximately 1% and is often a lifelong condition. It is the most common food-related cause of fatal allergic reactions in Western countries.

### SUMMARY

The present invention is based, at least in part, on the determination that administering a TLR4 agonist along with allergen, such as peanut allergen(s), sublingually can modulate pathogenic, allergen-specific immune responses. Specifically, glucopyranosyl lipid adjuvant (GLA) can be administered along with allergens, e.g., peanut proteins, in order to treat allergy in a patient, such as peanut allergy. The disclosed pharmaceutical compositions that include one or both of GLA and peanut proteins are sublingual immunotherapies (SLIT) intended for treatment of allergy, e.g., peanut allergy.

Accordingly, in one aspect, the present specification provides a pharmaceutical composition comprising a plurality of glucopyranosyl lipid adjuvant (GLA) particles (e.g., GLA liposomes, micelles, aggregates, or mixtures thereof), 1,2-dipalmitoyl-sn-glycero-3-phosphocholine (DPPC), and a therapeutically effective amount of allergen(s), e.g., peanut proteins, in a carrier (e.g., an aqueous carrier, i.e., a carrier in which water acts as a solvent), wherein the molar ratio of GLA to DPPC is in a range of about 1:1 to about 1:3 and at least some of the allergen(s), e.g., peanut proteins, are at least partially (e.g., completely) disposed within and/or on at least one of the lipid particles (e.g., liposomes) and/or free in the carrier (e.g., aqueous carrier).

In another aspect, the present specification provides a pharmaceutical composition comprising glucopyranosyl lipid adjuvant (GLA) and allergen(s), e.g., peanut proteins, wherein the pharmaceutical composition is in a solid or semi-solid dosage form.

In embodiments of the pharmaceutical compositions described herein, where an aqueous carrier is used, the aqueous carrier can include water. A composition comprising GLA can be in a form comprising a plurality of lipid particles, such as liposomes, micelles, and/or aggregates. The average lipid particle size in the plurality (e.g., the mean hydrodynamic diameter (Z-Average diameter) of the lipid particle) can be about 10 nm to about 2000 nm, e.g., about 16 nm to about 1800 nm, about 50 nm to about 1000 nm, about 80 nm to about 500 nm, e.g., about 90 nm, 100 nm, about 200 nm, about 300 nm or about 400 nm. A composition comprising GLA can be in a colloidal dispersion. The particle size can be measured using a Malvern Zetasizer, which provides the mean hydrodynamic diameter (Z-Average diameter) and the polydispersity index (PDI) of the particles. In some embodiments of any pharmaceutical compositions described herein, allergens(s) (e.g., peanut proteins and/or other allergen) may be included and completely enclosed within lipid particles or partially enclosed within lipid particles, or both, and/or disposed on lipid particle surface(s). In other embodiments, some allergens (e.g., peanut protein(s)) may be enclosed and/or partially enclosed by the lipid particles while others are free in the pharmaceutical composition and not enclosed and/or partially enclosed within lipid particles. In still other embodiments, substantially all allergens, e.g., peanut protein(s), are free in the pharmaceutical composition and not enclosed or partially enclosed within lipid particles.

The concentration of GLA in pharmaceutical compositions described herein can be, e.g., from about 0.2 μg/mL to about 5 mg/mL, e.g., about 2 μg/mL, 10 μg/mL, 20 μg/mL, 50 μg/mL, 100 μg/mL, 150 μg/mL, 200 μg/mL, 250 μg/mL, 300 μg/mL, 400 μg/mL, 500 μg/mL, 0.8 mg/mL, 1 mg/mL or about 1.6 mg/mL. For example, the concentration of GLA in the composition can be about 0.01 mg/mL to about 5 mg/mL, e.g., about 0.02 mg/mL to 0.2 mg/mL, or 0.16 mg/mL. Pharmaceutical compositions can include 0.001% to 0.1% DPPC, e.g., 0.01% to 0.05% DPPC, e.g., 0.02% to 0.03% DPPC. For example, a composition can contain about 0.010% DPPC, 0.015% DPPC, 0.020% DPPC, 0.025% DPPC, 0.030% DPPC, 0.035% DPPC, 0.040% DPPC, 0.045% DPPC, or about 0.050% DPPC. In some instances, the molar ratio of GLA to DPPC can be about 1:2. In one embodiment, a GLA composition includes a preservative, such as glycerol. For example, a GLA composition for use in a SLIT formulation can include 0.025% DPPC, 60% glycerol and water.

The concentration of peanut proteins in a pharmaceutical composition for sublingual administration can be about 2 μg/mL to about 25,600 μg/mL, e.g., about 2,000 μg/mL to about 7,000 μg/mL, e.g., about 5,000 μg/mL. In some embodiments, the concentration of peanut proteins in a pharmaceutical composition for sublingual administration is about 3 μg/mL, about 4 μg/mL, about 5 μg/mL, about 6 μg/mL, about 7 μg/mL, about 8 μg/mL, about 10 μg/mL, about 20 μg/mL, about 23 μg/mL, about 24 μg/mL, about 25 μg/mL, about 26 μg/mL, about 50 μg/mL, about 100 μg/mL, about 200 μg/mL, about 500 μg/mL, about 1 mg/mL, about 5 mg/mL, about 10 mg/mL, or about 20 mg/mL. In some embodiments, the concentration of peanut proteins in a pharmaceutical composition for sublingual administration is about 6.4 mg/mL or about 12.8 mg/mL. For example, the concentration of peanut proteins in a pharmaceutical composition described herein can be about 5 μg/mL to about 25,600 μg/mL or about 1,000 μg/mL to about 7,000 μg/mL.

Other allergens can be included in a pharmaceutical composition or method of making a pharmaceutical composition described herein, as an alternative, or in addition, to peanut allergens. For example, in some instances, a food allergen other than peanut allergen(s) can be included. Examples of such food allergens include milk allergen (e.g., whole milk or extract thereof, casein (e.g., alpha S1-casein), and/or beta-lactoglobulin), seafood allergen (e.g., allergens from vertebrates (e.g., salmon, cod, mackerel, sardines, herring, anchovies, tuna, trout, haddock, eel, and/or rays) and/or invertebrates (e.g., crustaceans (e.g., shrimp, crab, crayfish, and/or lobster allergen) and/or mollusks (e.g., clams, mussels, oysters, octopus, squid and/or scallops allergen)), egg allergen, mustard allergen, sesame allergen, soy allergen, wheat allergen (e.g., gluten), fruit allergen (such as Bet v 1 or homologues thereof, lipid transfer protein, and/or profilin, and/or allergens from strawberry, apple, avocado, blueberry, date, kiwi fruit, peach, raspberry, fig, grape, plum, cherry, grapefruit, and/or prune), vegetable allergen (e.g., alfalfa, cauliflower, cucumber, mushroom, radish, broad bean, eggplant, spinach, zucchini, broccoli, and/or pepper allergen) or tree nut allergen (e.g., walnut, almond, cashew, pistachio, and/or pecan allergen), to treat allergies to each of those foods. Alternatively or in addition, house dust mite (HDM) allergen, an aeroallergen (e.g., pollen allergen) and/or grass allergen can be included, e.g., for the treatment of an allergy, such as a seasonal allergy. Further, compositions that include such allergens can be used in the methods for treating allergy (i.e., an allergy to the allergen included in the pharmaceutical composition) described herein.

In some embodiments, the pharmaceutical composition can be formulated to deliver a dose of about 0.1 μg to about 80 μg GLA, e.g., 0.5 μg to 40 μg, e.g., 0.8 μg to 20 μg GLA. In some embodiments, the pharmaceutical sublingual formulation can include about 0.5 μg, about 1 μg, about 2 μg, about 5 μg, about 10 μg, about 20 μg, about 40 μg or about 50 μg GLA.

In some embodiments, a pharmaceutical composition described herein is formulated to deliver a dose of about 50 ng to about 30 grams or more peanut proteins, e.g. 100 ng to about 20 grams or more peanut proteins, about 300 ng to about 15 grams of more peanut proteins, about 500 ng to about 10 grams or more peanut proteins; 800 ng to about 9 grams or more peanut proteins; about 1000 ng to about 8.5 grams or more peanut protein; about 2 μg to about 8.3 grams or more peanut protein; e.g., about 5 μg, 10 μg, 20 μg, 40 μg, 80 μg, 100 μg, 160 μg, 250 μg, 320 μg, 500 μg, 640 μg, 1000 μg, 1280 μg, 2000 μg, 2560 μg, 3000 μg, 4000 μg, 5000 μg, 5120 μg, 6000 μg, 7000 μg, 10 mg, 20 mg, 40 mg, 50 mg, 100 mg, 500 mg, 1000, mg, 1500 mg, 2000 mg, or about 5000 mg, or more, peanut protein. The composition can be in a range from pH 4 to pH 8.5, e.g., about pH 4.5, pH 5.5, pH 7.5, or about pH 8.5. In some embodiments, the composition is at about pH 8.0, pH 8.1, pH 8.2 or about pH 8.3. The pharmaceutical composition can be formulated to deliver a dose of about 100 μg to about 8 grams or more peanut protein; e.g., about 500 μg to about 7.5 grams or more peanut protein.

Peanut proteins in the pharmaceutical compositions described herein can comprise or consist of one or more of peanut allergen components Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17, or any combination thereof. In some embodiments, the peanut allergens included in the pharmaceutical compositions consist of peanut allergen components Ara h1, Ara h2, Ara h3, and Ara h6. In other embodiments, the peanut allergens included in the pharmaceutical compositions consist of peanut allergen components Ara h1, Ara h2, and Ara h6. In still other embodiments, the peanut allergens included in the pharmaceutical compositions consist of peanut allergen components Ara h2 and Ara h6.

Pharmaceutical compositions described herein can be in the form of, e.g., a liquid, a semi-solid, a tablet (e.g., a fast disintegrating tablet (FDT), a gel capsule, a thin film, a sublingual drop, or a sublingual spray. Pharmaceutical compositions described herein may comprise a bioadhesive component. Pharmaceutical compositions can be in the form of a tablet or a multi-particulate granule or coated microsphere, e.g., packaged in a sachet form. FDTs described herein can include a superdisintegrant, e.g., cross-linked cellulose, cross-linked polyvinylpyrrolidone, cross-linked starch, or cross-linked alginic acid, or a mixture thereof. Compositions described herein can include a gelling agent, a binder, a glidant, an antiadherant, a flavoring agent, a sweetening agent, and/or a colorant, or any combination thereof. Thin films described herein can include, e.g., plasticized hydrocolloid. In certain thin films described herein, the GLA or allergen(s) (e.g., peanut proteins), or both, can be disposed within the thin film. In other thin films, the GLA or allergen(s) (e.g., peanut proteins), or both, are disposed on the surface of the thin film. In still other thin films, the GLA or allergen(s) (e.g., peanut proteins), or both, are disposed both within and on the surface of the thin film.

In another aspect, the specification provides a method of treating an allergy in a patient, comprising administering sublingually to a patient having an allergy a plurality of glucopyranosyl lipid adjuvant (GLA) particles and allergens (e.g., peanut proteins), in amounts effective to treat the allergy in the patient. The method can further comprise administering sublingually to the patient 1,2-dipalmitoyl-sn-glycero-3-phosphocholine (DPPC). In some embodiments, the plurality of GLA particles are formulated as a first pharmaceutical composition and the allergen(s) (e.g., peanut proteins) are formulated as a second, separate pharmaceutical composition. The first and second pharmaceutical compositions can be, e.g., administered simultaneously, or the first formulation can be administered to the patient prior to the second formulation, or the second formulation can be administered to the patient prior to the first formulation. In other embodiments, the GLA and the allergen(s) (e.g., peanut proteins) are formulated in a single pharmaceutical composition comprising a plurality of GLA particles and allergen(s) (e.g., peanut proteins) in an aqueous carrier. The pharmaceutical composition can further comprise 1,2-dipalmitoyl-sn-glycero-3-phosphocholine (DPPC), and in some instances the molar ratio of GLA to DPPC in the pharmaceutical composition can be in a range of about 1:1 to about 1:3.

In another aspect, the specification provides a method of treating an allergy in a patient, comprising administering sublingually to a patient having an allergy a pharmaceutical composition comprising glucopyranosyl lipid adjuvant (GLA) and allergen(s) (e.g., peanut proteins), wherein the pharmaceutical composition is in a solid or semi-solid dosage form. The pharmaceutical composition can be, e.g., a thin film or a tablet or capsule, as described herein.

In the methods of treating an allergy described herein, where peanut allergens are included in the treatment, the allergy can be a peanut allergy, an allergy to birch tree pollens, an allergy to peach and peach related fruits, or a combination thereof.

The methods of treating an allergy described herein can further include performing a basophil activation test on the patient prior to administering the treatment. Patients treated using the presently described methods can be, e.g., a human, e.g., an adult human (18 years or older) or juvenile human (17 years or younger). The patient can be an adolescent (age 12 years to 17 years), or a child (11 years of age or younger). The patient can be a child 4 to 11 years old.

In one embodiment, treatment of an allergic patient with a sublingual formulation described herein can prevent future allergic reactions of the patient upon exposure to peanut proteins. For example, in peanut allergy treatments, treatment of a patient can prevent or reduce the severity of an allergic reaction of a patient following exposure (such as by ingestion) of the patient to up to 20 grams or more of peanut proteins. For example, treatment of a patient with the adjuvanted sublingual formulation described herein can prevent or reduce the severity of an allergic reaction of a patient following exposure of the patient to 100 mg to 10 grams or more of peanut proteins, e.g, about 200 mg, 300 mg, 500 mg, 800 mg, 1000 mg, 2000 mg, 3000 mg, 4000 mg, or about 5000 mg of peanut proteins. Typically, treatment of a patient with the sublingual formulation results in the prevention of an anaphylactic reaction following accidental exposure to peanut proteins.

In still another aspect, the specification provides a method of making a pharmaceutical composition, the method comprising: co-dissolving glucopyranosyl lipid adjuvant (GLA) and 1,2-dipalmitoyl-sn-glycero-3-phosphocholine (DPPC) at a 1:2 molar ratio in chloroform to thereby form a GLA/DPPC mixture; adding allergen(s) (e.g., peanut proteins) to the GLA/DPPC mixture, to thereby form a GLA/DPPC/allergen(s) (e.g., peanut proteins) mixture; removing the chloroform from the GLA/DPPC/allergen(s) (e.g., peanut proteins) mixture; adding water to the GLA/DPPC/allergen(s) (e.g., peanut proteins) mixture; and agitating the GLA/DPPC/allergen(s) (e.g., peanut proteins) mixture, to thereby form a pharmaceutical composition.

In still another aspect, the specification provides a method of making a pharmaceutical composition, the method comprising co-dissolving glucopyranosyl lipid adjuvant (GLA) and 1,2-dipalmitoyl-sn-glycero-3-phosphocholine (DPPC) at a 1:2 molar ratio in chloroform to thereby form a GLA/DPPC mixture; removing the chloroform from the GLA/DPPC mixture; adding water to the GLA/DPPC mixture; agitating the GLA/DPPC mixture; and adding allergen(s) (e.g., peanut proteins) to the GLA/DPPC mixture, to thereby form a pharmaceutical composition.

In still another aspect, the specification provides a method of making a pharmaceutical composition, comprising mixing glucopyranosyl lipid adjuvant (GLA) and 1,2-dipalmitoyl-sn-glycero-3-phosphocholine (DPPC) at a 1:2 molar ratio in water, e.g., water that is heated, such as to a temperature of about 40° C. to 80° C., e.g., to 50° C. to 70° C., to thereby form a GLA/DPPC mixture. The GLA/DPPC mixture is then agitated at the elevated temperature (e.g., at about 70° C.); and then allergen(s) (e.g., peanut proteins) are added to the GLA/DPPC mixture, to thereby form a pharmaceutical composition.

In yet another aspect, the specification provides a method of making a pharmaceutical composition, comprising mixing glucopyranosyl lipid adjuvant (GLA) and a surfactant; adding water to the GLA/surfactant mixture; agitating the GLA/surfactant mixture; and adding allergen(s) (e.g., peanut proteins) to the GLA/surfactant mixture, to thereby form a pharmaceutical composition. The surfactant can be, e.g., Sodium Lauryl Sulfate, polysorbate-80, poloxamer 407 or poloxamer 188, or a combination of lecithin and taurocholate, or a combination thereof. In one example, the surfactant can be polysorbate-80, and the mixture can further include glycerol from 40% w/v to 80% w/v, e.g., glycerol at about 50% w/v, about 60% w/v, or about 70% w/v. In some instances, in the methods of making a pharmaceutical composition described herein, agitating can include sonication, microfluidization, high pressure homogenization, or mechanical agitation, such as with an Ultra-Turrax system, or any combination thereof. The methods can include compression molding the pharmaceutical composition to form a tablet. The methods can include lyophilizing or spray drying the pharmaceutical composition. The methods can include the steps of: forming a thin polymer film by a method selected from the group consisting of solvent casting, semisolid casting, hot melt extrusion, solid dispersion extrusion, and rolling; and disposing the pharmaceutical composition within thin film and/or on at least one surface of the thin film.

In any of the pharmaceutical compositions and methods of making the pharmaceutical compositions described herein, the peanut proteins can include one or more of the peanut allergen components Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17, or any combination thereof. In the pharmaceutical compositions and methods of making the pharmaceutical compositions described herein, the peanut proteins can consist of one or more of peanut allergen component Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17, or any combination thereof. For example, the peanut proteins can consist of peanut allergen components Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17. For example, the peanut proteins can consist of peanut allergen components Ara h1, Ara h2, Ara h3, and Ara h6. As another example, the peanut proteins can consist of peanut allergen components Ara h1, Ara h2, and Ara h6. As still another example, the peanut proteins can consist of peanut allergen components Ara h2 and Ara h6.

The methods of making the pharmaceutical compositions can further include prior to adding the peanut proteins to the GLA/DPPC mixture, performing a basophil activation test on the peanut proteins to measure the potency of the peanut proteins.

Also provided herein is the use of GLA and allergen(s), e.g., peanut allergen(s), together or separately in a sublingual formulation(s) for the treatment of allergy (e.g., peanut allergy) in a patient. Also provided herein is the use of at least GLA and/or allergen(s), e.g., peanut allergens(s), in the manufacture of a sublingual medicament for treatment or prevention of allergy, e.g., peanut allergy. The medicament can be used in a method for treating allergy in a patient. The medicament can be in any form as described herein, e.g., liquid, semisolid, or solid, compositions.

The terms “effective amount” and “effective to treat,” as used herein, refer to an amount or concentration of a composition described herein utilized for a period of time (including acute or chronic administration and periodic or continuous administration) that is effective within the context of its administration for causing an intended effect or physiological outcome. Effective amounts of a composition described herein for use in the present invention include, for example, amounts that prevent or reduce the intensity of an allergic reaction to ingestion or exposure to peanuts or products that include peanut-based ingredients, reduce the risk of such allergic reactions, reduce one or more symptoms of such allergic reactions, and/or improve the outcome of other peanut allergy treatments. An effective amount may be determined by one skilled in the art.

The terms “treat” and “treatment” are used herein to describe delaying the onset of, inhibiting, or alleviating the detrimental effects of a condition, e.g., peanut allergy or symptoms of allergic reaction to peanuts or products that include peanut-based ingredients.

The term “patient” is used throughout the specification to describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. The materials, methods, and examples are illustrative only and not intended to be limiting.

### DESCRIPTION OF DRAWINGS

FIG. 1A is a bar graph illustrating the effect of GLA and peanut proteins treatments (at varying concentration) on anaphylaxis score in mice following challenge with crude peanut extract (CPE). ††=p<0.001 compared to vehicle+control (CT) group; * p<0.05 compared to vehicle+CPE/CT group.

FIG. 1B is a bar graph illustrating the effect of GLA and peanut proteins treatments (at varying concentration) on anaphylaxis score in mice following challenge with crude peanut extract. The CPE-alone group is omitted from this presentation. ††=p<0.001 compared to vehicle+control (CT) group; * p<0.05 compared to vehicle+CPE/CT group.

FIG. 2A is a bar graph illustrating the effect of GLA and peanut proteins treatment (0.5 μg) on core body temperature drop in mice following challenge with crude peanut extract. †=p<0.05 compared to vehicle+control (CT) group.

FIG. 2B is a bar graph illustrating the effect of GLA and peanut proteins treatment (0.5 μg) on core body temperature drop in mice following challenge with crude peanut extract. The CPE-alone group is omitted from this presentation. †=p<0.05 compared to vehicle+control (CT) group.

FIG. 3 is a diagram of a typical peanut extraction process.

FIG. 4A is a histogram illustrating the effect of sublingual administration of GLA alone on antigen-specific T cell proliferation in draining cervical nodes.

FIG. 4B is a histogram illustrating the effect of sublingual administration of GLA alone on antigen-specific T cell proliferation in the spleen.

FIG. 4C is a histogram illustrating the effect of sublingual administration of Ova peptide alone on antigen-specific T cell proliferation in draining cervical lymph nodes.

FIG. 4D is a histogram illustrating the effect of sublingual administration of Ova peptide alone on antigen-specific T cell proliferation in the spleen.

FIG. 4E is a histogram illustrating the effect of sublingual co-administration of GLA and Ova peptide on antigen-specific T cell proliferation in draining cervical lymph nodes.

FIG. 4F is a histogram illustrating the effect of sublingual co-administration of GLA and Ova peptide on antigen-specific T cell proliferation in the spleen.

FIG. 5 is a bar graph illustrating that sublingual administration of GLA with antigen enhances antigen-specific T cell proliferation in the draining lymph nodes in mice.

### DETAILED DESCRIPTION

The present specification is based, at least in part, on formulations comprising allergens such as peanut proteins and the TLR-4 agonist GLA, which are particularly suitable for sublingual administration, e.g., for the treatment of peanut allergy. Accordingly, provided herein are compositions and methods for treating an allergy in a patient, e.g., a peanut allergy. In general, treatment can be accomplished by administering to a patient, via the sublingual route, GLA in combination with at least one type of allergen. The GLA and allergen(s), e.g., peanut allergen, can be administered to patients either in separate sublingual formulations or in a single sublingual formulation comprising both GLA and allergen, e.g., peanut allergen. The disclosed pharmaceutical formulations containing one or both of GLA and allergen(s), such as peanut allergen, are sublingual immunotherapies (SLIT) intended, e.g., for treatment of peanut allergy.

### Glucopyranosyl Lipid Adjuvant (GLA)

The presently described compositions comprise the adjuvant GLA, which is an art-known and commercially available compound. Typically, the GLA can be of formula (I):

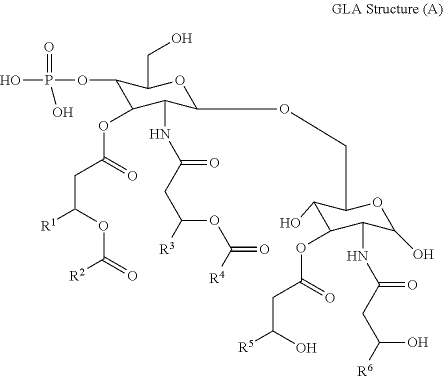

or a pharmaceutically acceptable salt thereof, where: R1, R3, R5 and R6 are C11-C20 alkyl; and R2 and R4 are C12-C20 alkyl; in a more specific embodiment, the GLA has the formula (I) set forth above wherein R1, R3, R5 and R6 are C11-14 alkyl; and R2 and R4 are C12-15 alkyl. In a further more specific embodiment, the GLA has the formula (I) set forth above wherein R1, R3, R5 and R6 are C11 alkyl, or undecyl; and R2 and R4 are C13 alkyl, or tridecyl. In yet a further specific embodiment, the GLA has the formula (I) set forth above wherein R1, R3, R5 and R6 are undecyl and R2 and R4 are tridecyl. GLA is known in the art and can be prepared by skilled practitioners and/or obtained from a variety of commercial sources.

For example, particularly useful in the present invention is GLA of formula (II), which can be obtained, e.g., from Avanti Polar Lipids, Inc.:

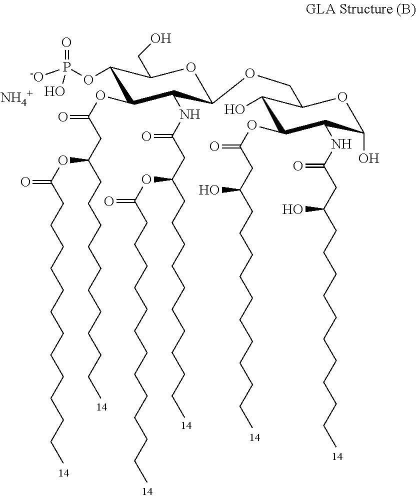

A pharmaceutically acceptable salt of GLA suitable for use in a sublingual formulation featured in the invention can be an aluminum salt or ammonium salt, or a benzathine salt. Other suitable salts include calcium, ethylene diamine, lysine, magnesium, meglumine, potassium, procaine, sodium, tromethamine and zinc. Anomeric variants of GLA can also be used in the sublingual formulations featured in the invention.

A GLA suitable for use in a sublingual formulation featured in the invention can be the free acid form of GLA with no counter ion salt (see Structure (A) above).

As used herein, “alkyl” means a straight chain or branched, noncyclic or cyclic, unsaturated or saturated aliphatic hydrocarbon containing from 1 to 20 carbon atoms, and in certain preferred embodiments containing from 11 to 20 carbon atoms. Representative saturated straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. For example, “C18-13 alkyl” and “C6-11 alkyl” mean an alkyl as defined above, containing from 8-13 or 6-11 carbon atoms, respectively.

As used herein, “acid functional group” means a functional group capable of donating a proton in aqueous media (i.e. a Brønsted-Lowry acid). After donating a proton, the acid functional group becomes a negatively charged species (i.e. the conjugate base of the acid functional group). Examples of acid functional groups include, but are not limited to: —OP(═O)(OH)₂ (phosphate), —OS(═O)(OH)₂ (sulfate), —OS(OH)₂ (sulfite), —OC(OH)₂ (carboxylate), —OC(═O)CH(NH₂)CH₂C(═O)OH (aspartate), —OC(═O)CH₂CH₂C(═O)OH (succinate), and —OC(═O)CH₂OP(═O)(OH)₂ (carboxymethylphosphate).

As used herein, “hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, that is, to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl,” respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Compounds of formula I and/or II may be obtained by synthetic methods known in the art, for example, the synthetic methodology disclosed in PCT International Publication No. WO 2009/035528. Skilled practitioners will also appreciate that GLA may be obtained commercially, e.g., from Avanti Polar Lipids, Inc.

### Allergens

As used herein, an “allergen” is any antigenic substance capable of producing an allergen-specific allergic reaction in a patient. An allergen can be, e.g., a protein, glycoprotein, carbohydrate, lipid, glycolipid, or other organic compound.

Some exemplary allergens useful in the present compositions and methods include (a) a full length antigen, (2) an immunogenic fragment of an antigen, (3) an immunogenic variant of a full length antigen or immunogenic fragment, (4) a chimeric fusion that includes portions from different polypeptides/antigens, and (5) a conjugate comprising two or more of such examples.

Peanut proteins contain multiple allergens, which are particularly useful in the compositions and methods described herein. Compositions described herein can include at least one type of peanut allergen. A peanut allergen used in a composition described herein can be, e.g., (a) a full length antigen, (2) an immunogenic fragment of the antigen, (3) an immunogenic variant of the full length antigen or immunogenic fragment, (4) a chimeric fusion that includes portions from different peanut polypeptides, and/or (5) a conjugate comprising two or more of such peanut allergens.

In some instances, the compositions include a single type of isolated peanut allergen, a mixture of isolated peanut allergens, or whole peanut extract, which is a mixture of peanut proteins that includes peanut allergens. As used herein, the term “isolated” means that a material is removed from its original environment (e.g., the natural environment if it is naturally occurring). With respect to individual types of peanut allergens, at least 17 of such allergens have been identified. Among them are Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17. Genbank Accession Numbers for the cDNA sequences of exemplary allergens include L34402.1 (Ara h1), AY007229.1 (Ara h2.0101), AY158467.1 (Ara h2.0201), AF093541.1 (Ara h3.0101), AF086821.1 (Ara h3.0201), AF059616 (Ara h5), AF092846.1 (Ara h6), AF091737.1 (Ara h7), EU046325.1 (Ara h7.0201), AY328088.1 (Ara h8.0101), EF436550.1 (Ara h8.0201), EU159429.1 (Ara h9.0101), and EU161278.1 (Ara h9.0201), AY722694.2 (Ara h10.0101), AY722695.1 (Ara h10.0201), DQ097716.1 (Ara h11), EY396089.1 (Ara h12), EY396019.1 (Ara h13), AF325917.1 (Ara h14.0101), AF325918 (Ara h14.0102), DQ368496 (Ara h14.0103), and AY722696 (Ara h15), respectively (see, e.g., Leon et al., The peanut allergy epidemic: allergen molecular characterisation and prospects for specific therapy. Expert Rev. Mol. Med. Vol. 9, Issue 1, January 2007; see also Arkwright et al., IgE Sensitization to the Nonspecific Lipid-Transfer Protein Ara h 9 and Peanut-Associated Bronchospasm, BioMed Research International, vol. 2013, Article ID 746507). Allergens Ara h1, 2, 3, 6, 8, and 9 are important markers of peanut sensitization and can be predictive of an allergic response. Ara h1, 2, and 3 are seed storage proteins. Ara h2 is a more important predictor of clinical peanut allergy than Ara h1 and 3, and is often associated with severe reactions. Ara h6 elicits antibodies that cross-react with Ara h2 and sensitization to Ara h6 and Ara h2 often occur together. Ara h8 is a pathogenesis-related (PR)-10 protein, and sensitization is often associated with mild, localized symptoms. Ara h8 cross-reacts with certain pollens (e.g., Birch and Birch-related tree pollen). Ara h9 is a lipid transfer protein, and sensitization can result in systemic reactions, including anaphylaxis. Patients with sensitivity to Ara h9 are often also sensitive to Ara h1-3. Ara h9 often cross-reacts with pitted fruits, such as peaches. Compositions described herein can comprise, for example, at least one type of peanut allergen selected from the group consisting of: Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16 and Ara h17, or an allergic reaction-inducing portion of Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17. It is contemplated, for example, that an extract of the whole peanut may be used, that a single peanut allergen or allergic reaction-inducing portion thereof may be used, and that any of the isolated peanut allergens and/or allergic reaction-inducing portions thereof can be used in any combination. For example, instead of whole peanut extract, the compositions may include any combination of at least 2, e.g., at least 3, 4, 5, 6, 7, 8, 9, 10, 11, 12, 13, 14, 15, 16, or 17, of Ara h1, Ara h2, Ara h3, Ara h4, Ara h5, Ara h6, Ara h7, Ara h8, Ara h9, Ara h10, Ara h 11, Ara h12, Ara h13, Ara h14, Ara h15, Ara h16 and Ara h17, or allergic reaction-inducing portion(s) of Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16 or Ara h17. For example, a composition described herein may include Ara h1, Ara h2, and Ara h6, or allergic reaction-inducing portion(s) thereof. As another example, the composition may include Ara h2, and Ara h6, or allergic reaction-inducing portion(s) thereof. As still another example, the composition may include Ara h1, Ara h2, Ara h3, and Ara h6, or allergic reaction-inducing portion(s) thereof. In yet another example, the composition may include Ara h12 and Ara h13, or allergic reaction-inducing portion(s) thereof. It is contemplated that combinations of any full-length allergen(s) and any allergic reaction-inducing portions of allergens may be used together.

In some instances, compositions described herein can include a mixture of full-length Ara h proteins, a mixture of allergic reaction-inducing portions of Ara h proteins (i.e., immunogenic fragments), or a combination of both full-length and allergic reaction-inducing portions of Ara h proteins. For example, an immunogenic fragment can include at least 5, e.g., at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50, 60, 70, 80, 90, or at least 100, or more, contiguous amino acids of the protein. Immunogenic fragments can be small, e.g. about 50 amino acids or less, or between about 6-10, 10-15, 15-20, 20-30, 30-40, 40-50, 50-60, 60-70, 70-80, 80-90, 90-100, or more contiguous amino acids. The immunogenic fragments may comprise a sufficient number of contiguous amino acids that form a linear epitope and/or may comprise a sufficient number of contiguous amino acids that permit the fragment to fold in the same (or sufficiently similar) three-dimensional conformation as the full-length polypeptide from which the fragment is derived to present a non-linear epitope or epitopes (also referred to in the art as conformational epitopes). Identifying an immunogenic region and/or epitope of an antigen can be readily determined by a person skilled in the art and/or by computer analysis and computer modeling, using methods and techniques that are routinely practiced by persons skilled in the art.

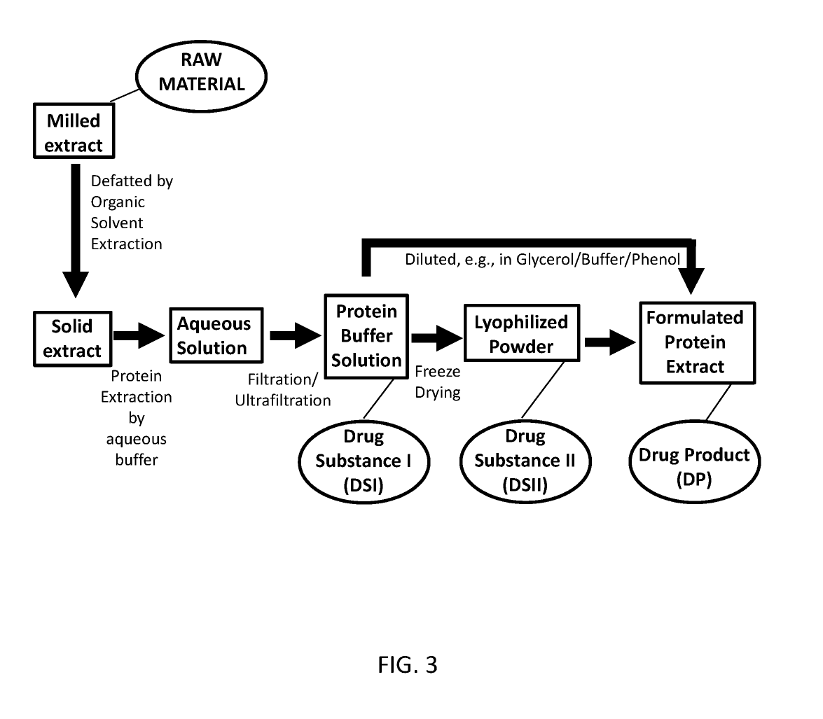

In some instances, compositions described herein can include a peanut extract, such as an extract made from roasted peanuts, or an extract made from raw (unroasted) peanuts. A pharmaceutically acceptable peanut extract is suitable for use in humans, and is referred to as a drug substance or a drug product (see, e.g., FIG. 3). A pharmaceutically acceptable peanut extract in the form of a drug substance can be further formulated for administration to humans in the form of a drug product. A peanut extract that is not processed to the stage that is acceptable for administration to humans is referred to herein as a “crude peanut extract.” Examples of a crude peanut extract include a peanut flour extract, peanut oil, and crude peanut powder extract, or liquid extract. A drug substance, which can also be a “new active substance”, can be a lyophilized peanut powder extract, a liquid peanut extract concentrate, or a diluted liquid peanut extract. A drug substance can also be a purified flour or oil extract.

A pharmaceutically acceptable peanut extract, which is a drug substance or a drug product, can be characterized in various ways, such as by measuring one or more of Ara h protein content, total protein content, lipid content, polysaccharide content metal content, aflatoxin content, microbial content, water content (particularly if the extract is lyophilized) and the like. For example, the extract can be characterized according to the amount of one or more of aflatoxin products B1, B2, G1 and G2, such as by HPTLC (high performance thin layer chromatography). Microbial content can include, for example, total aerobic microbial count (TAMC), total yeasts and molds count (TYMC) and measurement of specific microorganisms. In some embodiments, a pharmaceutically acceptable peanut extract can be characterized according to the amount of certain impurities, such as pesticides, solvents or other organic or inorganic materials contained in the extract.

In some embodiments, the extract is characterized by how much of a particular Ara h protein, or a combination of Ara h proteins, is contained in the extract. For example, a pharmaceutically acceptable peanut extract, or a crude protein extract (e.g., a crude protein extract with a known protein content) can be characterized by the percentage of 2 or more Ara h proteins in the extract, e.g., at least 3, 4, 5, 6, 7, 8, 9, 10, 11, 12, 13, 14, 15, 16, or 17, of Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17 protein. A peanut extract, such as a pharmaceutically acceptable extract can be characterized by the ratio of 2 or more Ara h proteins in the extract. For example, the Ara h2/Ara h6 ratio can range from about 3.0 to 1.0 (e.g., about 3.0, about 2.5, about 2.2, about 2.0, about 1.9, about 1.7, about 1.5, about 1.4, about 1.3, about 1.2, about 1.1, or about 1.0).

The total protein content of a pharmaceutically acceptable extract can be from about 12% to about 6% (e.g., about 11.5%, about 11%, about 10.5%, about 10%, about 9.5%, about 9%, about 8.5%, about 8%, about 7.5% or about 7%).

In one embodiment, the extract is characterized by the percent content of Ara h1, Ara h2, and Ara h6 content and total protein content. In another embodiment, the extract is characterized by the percent content of Ara h1, Ara h2, and Ara h6 content, the total protein content, and the lipid content. In yet another embodiment, the extract is characterized by at least (i) the percent content of Ara h1, Ara h2, and Ara h6 content, (ii) the total protein content, (iii) the lipid content, and (iv) the polysaccharide content.

The components of a peanut extract, such as a pharmaceutically acceptable peanut extract, can be assayed by methods known in the art. For example, the proteins present in the extract and the total protein content can be assayed by electrophoresis, such as by Bradford assay, SDS-PAGE and Western Blot, or Coomassie Stain; by ELISA assay, or by chromatographic methods, such as HPLC (high performance liquid chromatograph), or HPLC-UV or mass spectrometry methods, such as LC-MS (Liquid Chromatography-Mass Spectrometry). Such assays can further be used to identify the protein profile (e.g., ratio of Ara h proteins, such as Ara h1:Ara h2:Ara h6) of a peanut extract. The Ara h content of a peanut extract can be measured, for example, by ELISA, using specific antibodies (e.g., antibodies that specifically bind Ara h1, Ara h2 or Ara h6), or by LC-MS. Lipid content can be measured, for example, by HPTLC or HPLC-ESLD (Evaporating light scattering detector). Metal content can be measure, for example, by ICP-MS (inductively coupled plasma mass spectrometry).

A peanut extract, such as a pharmaceutically acceptable peanut extract, can be assessed qualitatively, such as by its color or clarity in solution.

In some embodiments a pharmaceutically acceptable peanut extract can be characterized functionally, such as by its ability to bind IgE and IgG in a biological sample (e.g., a serum sample) of a peanut allergic patient. In other embodiments, a pharmaceutically acceptable peanut extract can be characterized by a biological activity of the extract, such as by the ability of the extract to activate one or both of basophils or PBMCs (Peripheral Blood Mononuclear Cells) in a biological sample (e.g., a blood or serum sample) of a peanut allergic patient. A pharmaceutically acceptable peanut extract can be can be evaluated by measuring the potency of the extract, such as by using a basophil activation test as described in the examples below.

A pharmaceutically acceptable peanut extract can be characterized and established as an in house reference product (IHRP) to verify batch-to-batch consistencies. A pharmaceutically acceptable peanut extract can have a protein composition determined to be the same as an IHRP. A pharmaceutically acceptable peanut extract can have a protein content of 80% to 120% of the reference product, and individual allergens (e.g., certain Ara h proteins, such as Ara h1, Ara h2 and Ara h6 proteins) that are 50% to 200% of the amount identified in a reference product. In some instances, a lyophilized pharmaceutically acceptable peanut extract will have for example, not more than 5% water content. In some instances, a pharmaceutically acceptable peanut extract will meet the acceptance criteria set forth in the European Pharmacopoeia Monograph on Allergen Products (European Pharmacopoeia 7.0 01/2010:1063).

Allergens may be immunogenic variants of a one or more naturally occurring polypeptide antigens (e.g., an Ara h protein) that retain at least 90% amino acid identity over at least 10 contiguous amino acids of an antigen, or at least 85% amino acid identity over at least 15 contiguous amino acids of an antigen. Other examples include at least or about 70%, e.g., at least or about 75%, 80%, 85%, 90%, 91%, 92%, 93%, 94%, 95%, 96%, 97%, 98%, or at least or about 99% identity over at least 50 contiguous amino acids of the antigen, or at least or about 70%, e.g., at least 75%, 80%, 85%, 90%, 91%, 92%, 93%, 94%, 95%, 96%, 97%, 98%, or at least or about 99% identity over at least 100 contiguous amino acids of the antigen. These polypeptide immunogenic variants retain the ability to cross-react with immunoglobulins that are specific for the native antigen.

Variants can include at least one amino acid substitution, insertion, or deletion in an amino acid sequence. Conservative substitutions of amino acids are well known and may occur naturally in the polypeptide or may be introduced when the polypeptide is recombinantly produced. Amino acid substitutions, deletions, and additions may be introduced into a polypeptide using well-known and routinely practiced mutagenesis methods. Alternatively, random mutagenesis techniques, such as alanine scanning mutagenesis, error prone polymerase chain reaction mutagenesis, and oligonucleotide-directed mutagenesis may be used to prepare variants.

A variety of known criteria indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively.

A variety of methods are known in the art for recombinant production of polypeptide allergens. Methods that may be used for isolating and purifying a recombinant polypeptide, by way of example, may include obtaining supernatants from suitable host/vector systems that secrete the recombinant allergen or antigen into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. A variety of alternative purification methods are known in the art.

A composition comprising the allergen/antigen can in some instances be in the form of a composition comprising a recombinant expression vector that expresses the allergen/antigen. Thus, all references herein to a composition comprising an allergen or antigen apply equally to a composition comprising a viral vector carrying a nucleotide that encodes the allergen(s) or antigen(s).

Alternatively or in addition, a composition can include an allergen that is a chimeric fusion that includes multiple portions from different allergens, e.g., a fusion protein that includes a first full-length Ara h protein fused to a second full-length Ara h protein, a fragment of a first Ara h protein fused to a fragment of a second Ara h protein, or a first full-length Ara h protein fused to a fragment of a second Ara h protein. Methods of making fusion proteins are well known to those of ordinary skill in the art, and skilled practitioners will appreciate that many variations are possible, including fusion proteins that include whole or portions of at least 2, e.g., at least 3, 4, 5, 6, 7, 8, 9, 10, 11, 12, 13, 14, 15, 16, or at least 17, allergens, e.g., Ara h proteins.

Other allergens can be included in a pharmaceutical composition or method of making a pharmaceutical composition described herein, as an alternative, or in addition, to peanut allergens. For example, in some instances, a food allergen other than peanut allergen(s) can be included. Examples of such food allergens include milk allergen (e.g., whole milk or extract thereof, casein (e.g., alpha S1-casein), and/or beta-lactoglobulin), seafood allergen (e.g., allergens from vertebrates (e.g., salmon, cod, mackerel, sardines, herring, anchovies, tuna, trout, haddock, eel, and/or rays) and/or invertebrates (e.g., crustaceans (e.g., shrimp, crab, crayfish, and/or lobster allergen) and/or mollusks (e.g., clams, mussels, oysters, octopus, squid and/or scallops allergen)), egg allergen, mustard allergen, sesame allergen, soy allergen, wheat allergen (e.g., gluten), fruit allergen (such as Bet v 1 or homologues thereof, lipid transfer protein, and/or profilin, and/or allergens from strawberry, apple, avocado, blueberry, date, kiwi fruit, peach, raspberry, fig, grape, plum, cherry, grapefruit, and/or prune), vegetable allergen (e.g., alfalfa, cauliflower, cucumber, mushroom, radish, broad bean, eggplant, spinach, zucchini, broccoli, and/or pepper allergen) or tree nut allergen (e.g., walnut, almond, cashew, pistachio, and/or pecan allergen), to treat allergies to each of those foods. Alternatively or in addition, house dust mite (HDM) allergen, an aeroallergen (e.g., pollen allergen) and/or grass allergen can be included, e.g., for the treatment of an allergy, such as a seasonal allergy. Further, compositions that include such allergens alternative or in addition to peanut allergens can be used in the methods for treating allergy (i.e., an allergy to the allergen included in the pharmaceutical composition) described herein.

Preparation of Compositions for Sublingual Administration

Compositions that include GLA and separately allergens (e.g., separate GLA and peanut protein compositions), or a combination of both GLA and allergens (e.g., a combination of GLA and peanut proteins in a single composition), in a sublingual administration dosage form(s), can be prepared by a variety of methods. For example, a composition useful in the present invention is a composition comprising peanut allergens. Methods of extracting peanut proteins and producing allergen preparations are known to those of skill in the art. For example, a method of making a crude peanut extract is provided in U.S. Pat. No. 6,486,311. In some methods, peanuts are provided (e.g., from a commercial source), optionally roasted using methods known in the peanut-processing arts, and defatted with hexane (after roasting, if applicable) for 13 to 16 minutes at 163° C. to 177° C. The powdered crude peanut can then be extracted in 1 mol/L NaCl, 20 mmol/L sodium phosphate (pH 7.0) and 8 mol/L urea for 4 hours at 4° C. The extract can be clarified by centrifugation at 20,000 g for 60 minutes at 4° C. Another exemplary method is described in US. Patent Publication No. 2014/0363470, that includes grinding peanuts to peanut powder; incubating the peanuts in acetone for 30 minutes using 5 grams peanut powder per 50 mL acetone to provide defatted peanut powder, drying the defatted peanut powder, suspending the powder in a buffer with a pH between 7 and 9, and isolating the resulting supernatant to provide a whole peanut extract. Alternatively or in addition, peanut extract, peanut flour (comprising, e.g., grinded and defatted peanuts) and/or individual allergens can be obtained from a private, academic, government, or commercial source (e.g., Greer (North Carolina, US); Golden Peanut (Texas, US); Amanda Nut Processing Plant (Germany); INDOOR biotechnologies (Virginia, US)).

A composition described herein comprising peanut proteins (whether alone or in combination with GLA) can include peanut proteins in the range of about 0.3 μg/100 ml to about 26,000 μg/ml, e.g., 0.5 μg/100 ml to 25,600 μg/ml, e.g., 5 μg/100 ml to about 10,000 μg/ml, e.g., about 3 μg/ml to about 5,000 μg/ml. For example, compositions may include peanut proteins in the range of about 100 μg/ml to about 25,600 μg/ml, or about 2000 μg/ml to about 10,000 μg/ml. A composition described herein can include peanut proteins of at least or about 1 μg/ml, e.g., at least or about 3 μg/ml, 10 μg/ml, 20 μg/ml, 24 μg/ml, 25 μg/ml, 26 μg/ml, 50 μg/ml, 100 μg/ml, 200 μg/ml, 400 μg/ml, 500 μg/ml, 1000 μg/ml, 2000 μg/ml, 3000 μg/ml, 4000 μg/ml, 5000 μg/ml, 6000 μg/ml, 6400 μg/ml, 6500 μg/ml, 6600 μg/ml, 7000 μg/ml, 8000 μg/ml, 9000 μg/ml, 10,000 μg/ml, 15,000 μg/ml, 20,000 μg/ml, 25,000 μg/ml, or 26,000 μg/ml.

A composition comprising peanut proteins can include a salt, such as sodium chloride, or sodium citrate, in an amount of 0.05% to 1%, e.g., 0.1% to 0.5%, e.g., 0.2% to 0.3%, e.g., 0.25%. A composition comprising peanut proteins can contain a preservative, such as glycerol, in an amount of 30% glycerol to 80% glycerol, e.g., 40% glycerol to 60% glycerol, e.g., 45% glycerol, 50% glycerol or 55% glycerol. A composition comprising peanut proteins can be at neutral or basic pH, e.g., at pH 7 to pH 9, e.g., pH 7.5, pH 8.0, pH 8.2, pH 8.5.

In one embodiment, a composition comprising peanut proteins for sublingual administration (with or without GLA) comprises 0.25% NaCl, 0.27% NaHCO₃, 60% glycerol, and water. In one embodiment, the composition has a pH of about 8.2.

A composition comprising GLA can be prepared in any number of art-known ways. In one exemplary method, GLA can be synthesized or obtained from commercial suppliers (e.g., Avista Pharma Solutions (Durham, N.C.), Avanti Polar Lipids (Alabaster, Ala.), Immune Design Corporation (Seattle, Wash.)). The GLA can be combined with a lipid, such as 1,2-dipalmitoyl-sn-glycero-3-phosphocholine (DPPC; commercially available, e.g., from Avanti Polar Lipids, Inc.), in a molar ratio of about 1:2 (GLA:DPPC), and optionally dissolved in chloroform by vortexing or by other art-known mixing means. The chloroform can then be evaporated (e.g., under vacuum) and the resulting thin film solid mixture can be rehydrated and diluted, e.g., in water. The mixture can then be agitated (e.g., by one or more of mechanical agitation, such as by Ultra-Turrax® (IKA®, Wilmington N.C.), by BecoMix (A. Berents GmbH & Co. KG, Stuhr, Germany), or by sonication, microfluidization or high pressure homogenization, or any combination thereof, such as to generate nanoparticles) and filtered. Sonication of the formulation during the methods yields a formulation that includes GLA lipid particles, such as liposomes, micelles, and/or aggregates, or combinations thereof. In some instances, the final, average lipid particle size is less than 100 nm. In some instances, the final, average size of the lipid particles in the formulations produced by methods described herein is about 10 nm to about 2000 nm, e.g., about 16 nm to about 1800 nm, about 50 nm to about 1000 nm, about 80 to about 500 nm, about 85 nm to about 300 nm, about 95 nm to about 200 nm, about 90 nm to about 100 nm. In some instance, the final, average lipid particle size is less than 500 nm.

A composition comprising GLA can contain DPPC at a concentration of, e.g., 0.001% to 0.1% DPPC, e.g., 0.01% to 0.05% DPPC, e.g., 0.02% to 0.03% DPPC, e.g., 0.025% DPPC. A composition comprising GLA can contain a preservative, such as glycerol, in an amount of 30% glycerol to 80% glycerol, e.g., 40% glycerol to 60% glycerol, e.g., 45% glycerol, 50% glycerol or 55% glycerol. A composition comprising peanut proteins can be at neutral or basic pH, e.g., at pH 7 to pH 9, e.g., pH 7.5, pH 8.0, pH 8.2, pH 8.5.

In one embodiment, a composition comprising GLA (with or without peanut allergen) comprises 0.025% w/v DPPC, 60% w/v glycerol and water. The formulation can be comprised in water. In some embodiments, the formulation can be filtered.

In some instances, it may be beneficial to not use chloroform in the preparation methods. Accordingly, another exemplary method omits using chloroform in the procedure. For example, GLA and DPPC can be combined in a molar ratio of 1:2. Warm purified water (e.g., at 50° C. to 75° C.) is added and the mixture is agitated, such as by mechanical agitation or sonication and filtered. For example, in one embodiment, GLA and 0.025% DPPC are combined. Warm purified water (e.g., at 70° C.) is added, and the mixture is mechanically agitated using Ultra-Turrax. In some embodiments, the mixture is further filtered.

Importantly, skilled practitioners will appreciate that lipids or surfactants alternative or in addition to DPPC can be used. Exemplary alternative lipids or surfactants include, e.g., Sodium Lauryl Sulfate; Polyoxyethylene (20) sorbitan monooleate (also known as polysorbate-20 and Tween-80); poloxamer (also known under the trade names Synperonics, Pluronics, and Kolliphor), e.g., poloxamer 407 or polxamer 188; camphor; Tween-20; or mixtures thereof. In one instance, the preparation will utilize polysorbate (e.g., polysorbate-80) with 50% or 60% glycerol, which may be useful to maintain a preservative free formulation. In one example, the sublingual formulation will include one or both of lecithin and taurocholate.

In one embodiment, the sublingual formulation is free of preservatives.

Of particular usefulness in the presently described methods is Tween-80 (e.g., at 0.5%-10% of the formulation) which, when used with GLA, may reduce (or eliminate) the need to sonicate the GLA solution to form a nanosuspension, which is particularly useful, e.g., during scaled-up processes.

The concentration of GLA in a composition produced by methods described herein can be in a range of about 0.002 mg/mL to about 8.0 mg/ml, about 0.005 mg/mL to about 7.0 mg/ml, about 0.01 mg/mL to about 6.0 mg/ml, about 0.02 mg/mL to about 5.5 mg/ml, e.g., about 0.05 mg/ml to about 5.0 mg/ml, about 0.1 mg/ml to about 4.0 mg/ml, about 0.2 mg/ml to about 3.5 mg/ml, about 0.3 mg/ml to about 3 mg/ml, about 0.4 mg/ml to about 2.5 mg/ml, about 0.5 to about 2.0 mg/ml, or about 0.6 to about 2 mg/ml. For example, the GLA concentration can be about 0.02 mg/mL, about 0.1 mg/mL, about 0.8 mg/ml or about 1.6 mg/ml. Further, in the methods of preparing the GLA described herein, the molar ratio of GLA to DPPC can be in a range of from about 1:1 to about 1:3, e.g., about 1:2. In some instances, in addition to GLA, at least one other type of lipid and/or adjuvant (e.g., at least 2, at least 3, or at least 4 types of lipid and/or at least 2, at least 3, or at least 4 types of adjuvant) can be included in the compositions described herein. Alternatively or in addition, in some instances, in addition to DPPC, at least one other type of surfactant (e.g., at least 2, at least 3, or at least 4 types of surfactants) can be included in the compositions described herein.

A composition comprising both GLA and peanut proteins can be prepared by mixing an aqueous solution of the peanut proteins described above (e.g., whole peanut extract or individual allergens described herein) with GLA and the surfactant (e.g., DPPC). Alternatively, GLA may be co-precipitated with the antigen. In one example, the method includes co-dissolving glucopyranosyl lipid adjuvant (GLA) and 1,2-dipalmitoyl-sn-glycero-3-phosphocholine (DPPC) at a 1:2 molar ratio in a solvent, such as chloroform, to form a GLA/DPPC mixture and then adding peanut allergen, such as in the form of peanut proteins, to the mixture. The solvent, e.g., chloroform, is then removed from the GLA/DPPC/peanut proteins mixture, e.g., under vacuum. Water is then added to the GLA/DPPC/peanut proteins mixture to rehydrate the mixture and then the mixture is agitated, e.g., via sonication and/or microfluidization. Another exemplary method includes co-dissolving glucopyranosyl lipid adjuvant (GLA) and 1,2-dipalmitoyl-sn-glycero-3-phosphocholine (DPPC) at a 1:2 molar ratio in chloroform to form a GLA/DPPC mixture. The chloroform is then removed from the GLA/DPPC mixture, e.g., under vacuum. Water is then added to the GLA/DPPC mixture and then the mixture is agitated (e.g., using mechanical agitation, such as by Ultra-Turrax, or sonication and/or microfluidization). Peanut protein extract is then added to the formulation in amounts appropriate to achieve the desired concentration of proteins.

In another exemplary method, GLA and DPPC are co-dissolved at a 1:2 molar ratio in chloroform to form a GLA/DPPC mixture. The chloroform is then removed from the GLA/DPPC mixture, e.g., under vacuum. The mixture is then spray-dried to form a solid particle of GLA/DPPC. The peanut proteins are then layered on the surface of the GLA/DPPC particle by a fluid bed coating process. The final composition is a dry powder containing GLA and peanut proteins.

Alternatively, skilled practitioners will appreciate that separate compositions of GLA and peanut proteins can be created as described herein and mixed together shortly before administration to a patient.

The compositions may conveniently be formulated with a pharmaceutically acceptable diluent, carrier or excipient suitable for administration. Details of pharmaceutical excipients may be found in “Handbook of Pharmaceutical Excipients”, 7th Ed. (2012), The Pharmaceutical Press, London, Editors: Rowe et al. Suitable physiologically acceptable carriers and diluents include, e.g., sterile water or 5% dextrose water solution. Sterile saline and/or phosphate buffered saline at physiological pH may be used. Preservatives, stabilizers, dyes and/or flavoring agents may be provided in the pharmaceutical compositions described herein. For example, sodium benzoate, sorbic acid and esters of p-hydroxybenzoic acid may be added as preservatives. Antioxidants and/or suspending agents may be included.

Sublingual administration of the presently described compositions is particularly useful in the present invention. A gel or other viscous formulation may be useful due to increased antigen contact with the sublingual surface. Skilled practitioners will appreciate that a formulation need not physically or chemically bind to the mucosal tissue.

Formulations suitable for sublingual administration may include aqueous and non-aqueous sterile solutions. Such formulations may include anti-oxidants, buffers, bacteriostatic compounds and solutes that render the formulation isotonic with the bodily fluid, e.g., the mucus, of the individual. The aqueous and non-aqueous sterile suspensions may include suspending agents or thickening agents.

Specifically, compositions described herein, including compositions comprising GLA alone or antigen alone, and compositions comprising both GLA and antigen, can be incorporated into forms for sublingual delivery, such as gels, capsules, gel capsules, lozenges, tablets, sprays, drops, strips, or films, or disposed in devices such patches (e.g., for extended release delivery) or pumps or sprayers. The compositions can be administered by a metered dosing pump, for example. The compositions can alternatively be incorporated into multi-particulate granules or microspheres and optionally packaged in a container, such as such as sachet.

By way of example, a spray or drop formulation can be prepared for delivering peanut proteins and GLA, either in separate formulations or as a combined formulation. A spray or drop formulation useful in the present invention may include about 0.5% NaCl, 0.54% Na-bicarbonate, and 50% glycerol. Alternatively, a spray or drop formulation useful in the present invention may include about 0.5% NaCl, 0.54% Na-bicarbonate, and 60% glycerol. The formulation can have a pH of about 4.0 to about 8.4 (e.g., pH 5.0, pH 5.5, pH 6.0, pH 6.8, pH 7.0, pH 7.5 or pH 8.0). One exemplary spray or drop formulation includes a flavoring, such as a citric flavoring (e.g., orange or cherry), and has pH 4 to 4.5.

In another example, the compositions may be in the form of salves, pastes, gels, solutions, powders and the like. Gels may be formulated using carbopol, also known as carbomer, a carboxyvinyl polymer, or a cellulose-based thickening agent such as hydroxyethyl cellulose, hydroxypropyl cellulose or hydroxypropyl methylcellulose, carboxymethylcellulose calcium, carboxymethylcellulose sodium, ethyl cellulose, methylcellulose. Gels may also be conveniently formulated using: acacia, alginic acid, bentonite, carrageenan, cetostearyl alcohol, gelatin, guar gum, magnesium aluminium silicate, maltodextrin, polyvinyl alcohol, propylene carbonate, propylene glycol alginate, colloidal silicon dioxide, sodium alginate, tragacanth, and/or xanthan gum. Particularly useful are carbopol and the cellulose-based agents.

A pharmaceutical composition formulated as a sublingual tablet is particularly useful in the methods described herein. Sublingual tablets are placed under the tongue and are usually small and flat, compressed lightly to yield a soft tablet. The tablet should dissolve easily allowing the composition to be absorbed quickly. Various techniques can be used to formulate rapidly disintegrating or dissolving tablets (see, e.g., Fu et al., Crit Rev Ther Drug Carrier Syst. 2004; 21: 433-476). Direct compression is one of these techniques, which can involve incorporation of a superdisintegrant into the formulation or the use of highly water-soluble excipients to achieve fast tablet disintegration. Direct compression does not require the use of water or heat during the formulation procedure and is an ideal method for moisture and heat-labile medications. Superdisintegrants include, for example, cross-linked cellulose, cross-linked polyvinylpyrrolidone, cross-linked starch, and cross-linked alginic acid. A sublingual tablet can include, for example, one or more of a binding agent, such as gelatin, a microcrystalline cellulose, sucrose with dextrins, polyvidone, tragacanth, acacia, starch, or methylcellulose; a matrix supporting/disintegrating agent such as mannitol, L-alanine, cornstarch, alginic acid, cellulose or a cellulose derivative, povidone or sodium crosscarmelose; a lubricant, such as stearic acid, stearate (e.g., magnesium stearate), sodium stearyl fumarate, or talc; a buffer; a surface active agent; an anti-adhering agent selected from colloidal silicon dioxide, calcium sulfate, calcium chloride, talc, corn starch; polar and non-polar dissolving agents selected from water, ethyl alcohol, acetone, isopropyl myristate, polyoxypropylenes, propylene glycol, polyethylene glycol, glycerol, 70% sorbitol, polyethylene glycols, mineral oil, petrolatum, lanoline, vegetable waxes, animal waxes, vegetable oils such as olive oil, cottonseed oil, corn oil, or a mixture thereof; and a sweetener (e.g., dextrose, sucralose, stevia, aspartame, acesulfame potassium, fructose, glucose, mannitol, sorbitol, sugar, and sucrose) or other flavoring agent (e.g., chocolate, menthol, vanillin, cinnamon, sorbitol, citric acid, cherry flavor, orange flavor, pineapple flavor, peach flavor, grape flavor, or berry flavor, such as strawberry or blueberry flavor), In one embodiment, the flavoring is a citric flavor such as SyrSpend cherry, SyrSpend grape, or Ora-Sweet cherry. The flavoring agent can be present in a ratio from 0.0001% to 5.0%.

Exemplary tablets include fast disintegrating sublingual tablets (FDT) (which can be lyophilisates), bioadhesive sublingual tablets, compressed tablets (ODT—orally disintegrating tablets), and lipid matrix sublingual tablets. Exemplary methods of making such tablets are described, e.g., in Nibha et al., International Journal of Research in Pharmaceutical and Biomedical Sciences, p. 913-923, vol. 3 (2) April-June 2012. A sublingual tablet can include, for example, peanut extract (from roasted or raw peanuts), GLA, corn starch, and lactose monohydrate magnesium stearate. A sublingual tablet can include, as another example, peanut extract (from roasted or raw peanuts), GLA, mannitol, microcrystalline cellulose, a sodium carboxymethylcellulose, such as croscarmellose sodium, colloidal anhydrous silica, magnesium stearate and lactose monohydrate. Still another example of a sublingual tablet is one that includes peanut extract (from roasted or raw peanuts), GLA, gelatin, mannitol, and sodium hydroxide. In some instances, sublingual tablet formulations can contain one of, but not both of, GLA or peanut extract.

Also useful are thin films or strips. A thin film or strip dissolves when in contact with liquid, e.g., the saliva of a patient, and is placed under the tongue for an appropriate amount of time to allow the film or strip to dissolve. Fast-dissolving films can be made of plasticized hydrocolloids. Films are typically stable to moisture, are flexible, and may be formulated to resist sticking to packaging materials and fingers. Exemplary methods of making thin films or strips for sublingual administration include solvent casting, semisolid casting, hot melt extrusion, solid dispersion extrusion, and/or rolling (see, e.g., Nibha et al., International Journal of Research in Pharmaceutical and Biomedical Sciences, p. 913-923, vol. 3 (2) April-June 2012). Skilled practitioners will appreciate that thin films or strips of the present invention can take a number of different configurations. For example, the GLA or peanut proteins (e.g., peanut extract made from roasted or raw peanuts), or both, can be disposed within the thin film. Alternatively, the GLA or peanut proteins (e.g., peanut extract made from roasted or raw peanuts), or both, can be disposed on the surface of the thin film. Alternatively, GLA or peanut proteins (e.g., peanut extract made from roasted or raw peanuts), or both, can be disposed both within and on the surface of the thin film.

A thin film or strip for sublingual administration typically contains one or more of, e.g. a polymer, such as pullulan or microcrystalline cellulose with maltodextrin; a plasticizer, such as glycerol, propylene glycol, a low molecular weight polyethylene glycol, a phthalate derivative, such as dimethyl, diethyl, or dibutyl phthalate, a citrate derivative with as tributyl, triethyl, acetyl citrate, triacetin or castor oil; a sweetener; a coloring agent, and thickening and stabilizing agents.

Of particular usefulness are compounds that can be added to the formulations to enhance sticking or retention of the formulation under the tongue of a patient, i.e., a mucoadhesive agent. Exemplary mucoadhesive agents include chitosan, hyaluronate, alginate, gelatin, collagen, poly(acrylic acid), poly(methacrylic acid), poly(L-lysine), poly(ethyleneimine), poly(ethylene oxide), poly(2-hydroxyethyl methacrylate), and derivatives or copolymers thereof, and any combination thereof. Skilled practitioners will appreciate that a mucoadhesive can be added to any of the formulations or dosage forms described herein.

There are various commercial sources for manufacturing solid dosage forms, such as Catalent®, which uses Zydis® technology; Colorcon®, which produces Suglets® Sugar Spheres; Umang Pharmatech, which produces Sprayspheres™ microcrystalline cellulose (MCC) pellets.

The formulations may be presented in unit-dose or multi-dose containers, for example, sealed ampoules and vials and may be stored in a freeze-dried condition requiring only the addition of the sterile liquid carrier immediately prior to use.

### Treatments and Administration of Compositions

While not necessarily required for carrying out treatments described herein, patients may be diagnosed with peanut allergy and/or monitored according to standard clinical criteria prior to and/or during treatment. Standard clinical criteria may include, for example, a history of a type-1 hypersensitivity reaction that is temporally related to peanut ingestion (e.g. hives, swelling, wheezing, abdominal pain, vomiting, breathlessness). The presence of peanut-specific IgE by positive skin prick test (wheal diameter≧3 mm) or ImmunoCap serum IgE>0.35 kU/1, may also be indicative of peanut allergy. A patient's blood can be assayed, e.g., using the basophil activation test described in detail in the Examples section, below.

The compositions are administered in a manner compatible with the dosage formulation, and in such amount as will be prophylactically and/or therapeutically effective. The quantity to be administered depends on the patient to be treated, the capacity of the patient's immune system to synthesize antibodies, and the degree of protection desired. In some instances a patient can be treated with a series of administrations which will include a rising peanut proteins dose regime. The precise amounts of active ingredients administered may depend on the judgment of the practitioner and may be peculiar to each individual patient. The composition may be given in a single dose schedule, or in a multiple dose schedule.

In one exemplary dosage regimen, a composition comprising GLA, such as a solid or liquid composition comprising GLA, is administered first, followed by administration of a composition (e.g., a solid or liquid composition) comprising allergen, e.g., peanut allergen(s). In another exemplary dosage regimen, both the GLA and peanut allergen(s) are administered at approximately the same time. In one regimen, a GLA composition that includes GLA (e.g., a liquid formulation at 1 μg/50 μl or 10 μg/50 μl, or in a range between these two values, inclusive) and DPPC (or GLA, DPPC and glycerol, or GLA, DPPC, and Tween 80) is provided. An amount of the GLA composition is administered sublingually, i.e., under the tongue of the patient (e.g., about 50 μl to about 500 μl of a liquid formulation). Following and/or during administration of the GLA composition to the patient, an amount of a peanut proteins composition (e.g., about 50 μl to about 200 μl of a liquid formulation having protein concentration of about 6 μg/ml to about 25.6 mg/ml, inclusive, e.g., a protein concentration of 6 μg/ml or 25.6 mg/ml) is administered sublingually, under the tongue of the patient. Here, the peanut protein composition can include, e.g., 50% glycerol or 60% glycerol or 70% glycerol, and, optionally, 0.4% phenol as a preservative, 0.2% to 0.5% NaCl, 0.20% to 0.45% sodium bicarbonate, and 50% glycerol, at a pH of between 6.8 to 8.4. In one embodiment, the peanut protein composition includes, e.g., 60% glycerol, 0.25% NaCl, 0.27% sodium bicarbonate, 60% glycerol, and water at a pH of about 8.2.

In another exemplary regimen, a composition that includes GLA (e.g., a solid formulation) and a compound to aid the solubilization of GLA, such as DPPC or a polysorbate, such as PS80 or PS20, is provided. The GLA composition can include one or more of DPPC, a polysorbate, glycerol, or Tween (e.g., Tween 80). An amount of the GLA composition is administered sublingually, i.e., under the tongue of the patient. A solid formulation, in the form of a capsule or tablet, for example, can contain a therapeutically effective amount of GLA, such as 0.5 μg, 1 μg, 1.5 μg, 2 μg, 3 μg, 5 μg, 7 μg, 10 μg, 12 μg, 15 μg, 20 μg or more of GLA, for sublingual administration.

In some instances, a solid formulation comprising GLA and separate solid formulation comprising peanut protein are administered to the patient at about the same time, e.g., by placing the solid formulations together under the tongue or on opposite sides under the tongue of the patient. In other instances, a single solid formulation comprising both GLA and peanut protein is placed under the tongue of the patient. In other instances, a liquid formulation comprising GLA and a separate liquid formulation comprising peanut protein are administered to the patient at about the same time, e.g., by placing the liquid formulations together under the tongue or on opposite sides under the tongue of the patient. In still other instances, a single liquid formulation comprising both GLA and peanut protein is placed under the tongue of the patient. In yet other instances, one of each type of composition, i.e., one solid (comprising GLA or peanut protein) and one liquid (comprising GLA or peanut protein, i.e., whichever component is not comprised within the solid composition), can be administered to the patient.

The frequency with which a patient is dosed with the compositions can vary depending upon the needs of the patient and the level of protection required. For example, in some instances, only a single dose on a single day of each composition (i.e., the GLA composition and the peanut proteins composition) is administered to a patient. In other instances, the patient can be dosed more than once on a single day, such as twice, thrice, or more times in a single day. In still other instances, the patient can be dosed once a day for multiple days (e.g., for at least 2, e.g., at least 3, 4, 5, 6, or 7 days), or for several weeks (e.g., at least 2, e.g., at least 3, 4, 5, 6, 7, 8, or more than 8, weeks) or for several months (e.g., at least 2, e.g., at least 3, 4, 5, 6, or more than 6, months), or more than once each day (e.g., at least twice, or at least thrice), for multiple days (e.g., for at least 2, e.g., at least 3, 4, 5, 6, or 7 days), or for several weeks (e.g., at least 2, e.g., at least 3, 4, 5, 6, 7, 8, or more than 8, weeks) or for several months (e.g., at least 2, e.g., at least 3, 4, 5, 6, or more than 6, months, up to multiple years). Skilled practitioners will appreciate that the methods can be adapted for delivery of a combined GLA/peanut protein formulation to patients. Such a formulation may take the form of a liquid combined GLA/peanut protein formulation or a solid, dissolvable tablet combined GLA/peanut protein formulation.

Subjects suitable for administration of the GLA and peanut SLIT formulations featured herein include children 1 year old and older, e.g., children 4 years old and older. Patients suitable for administration of the GLA and peanut SLIT formulations featured herein include children 2 to 12 years of age, e.g., 4 to 10 years of age, adolescents 13 to 20 years of age, and adults 21 years of age and older.

The efficacy of the peanut protein/GLA combination sublingual therapy described herein can be measured by examining expression of certain markers in the patient, e.g., markers of the immune system, such as cytokines and interleukins. For example, the patient can be assessed for the effect of the formulation on expression of a Th1 cytokine, such as IL-1β, IL-6, or IFN-γ, IFN-β or a Th2 cytokine such as IL-4, IL-5, IL-10, or IL-13. In some embodiments, the patient is assessed for an effect on expression of IL-10, IL-7, IL-8, IL-2, IL-12, IL-17, GM-CSF, CRP (C-Reactive Protein), fibrinogen, RSAD2, IFIT1B, TLR4, TNFα, TNFγ, CXCL2, CXCL10, CCL4, CCL7, CD154, a Type I interferon and/or TGFβ in a biological sample, such as a saliva or blood or serum sample, or a sample (e.g., a swab or a biopsy) from the sublingual mucosa. For example, administration of a formulation comprising GLA and antigen can prevent upregulation of CD154 or IL-13 expression following exposure to antigen, and can maintain or increase IL-10 expression levels, or prevent a decrease in IL-10 expression. The effect on biomarker expression can be assayed in peripheral blood mononuclear cells (PBMCs) isolated from the patient. Expression can be monitored by assaying for changes in mRNA or protein levels. The patient can also be monitored for upregulation of dendritic cells (DCs) or macrophage cell surface expression of one of more of CD40, CD80, CD83, CD86, and MHC II. For example, the patient can be monitored for upregulation of macrophage expression of CD80 and CD86. The patient can also be monitored for the expression of one or more of salivary peanut allergen-specific IgA, peanut allergen-specific IgE, peanut allergen-specific IgG, or allergen-specific Ig4. In one embodiment, the patient is assessed for the effect of the formulation on IL-6 levels in a biological sample, such as in a sample from the sublingual mucosa of the patient. Typically, administration of a GLA/peanut allergen formulation will cause IL-6 levels to rise at a dose-dependent manner at the site of sublingual administration.

In some embodiments, a basophil activation test will be used to measure the effect of the GLA/peanut allergen sublingual therapy. For example, the basophil activation test can be used to sample biomarker expression in a blood sample from the patient. Exemplary biomarkers include IgE, CD203c, HLS-DR, CD123, CD63, and Lin. Typically CD203c, CD63 and basophil degranulation (e.g., histamine and beta hexosaminidase release) will be decreased, in a blood sample from a patient treated with the GLA/peanut allergen sample, as compared to levels prior to treatment. Also, peanut allergen-induced memory T cell activation will be decreased, which will be evident by a decrease in allergen-induced proliferation, and IL-2 and IL-13 expression, and an increase in IFNγ and IL-10 expression. The patient can also be monitored for an expected increase in T regulatory cell activity, which is typically evidenced by an increase in FoxP3 positive T cells, IL-10 expression, and increased epigenetic changes at the FoxP3 locus of regulatory T cells. The patient can also be monitored for an increase in peanut allergen-specific IgGs, including IgG1, IgG2a, IgG2c, and IgG4.

Further, combination treatments are contemplated by the present invention. For example, a skilled practitioner will appreciate that the treatment methods described herein can be administered to a patient in conjunction with other, art-known treatments for allergy e.g., peanut allergy. Such combination treatments may include known treatments for the allergy itself, and/or one or more symptoms of allergy in the patient.

Further, since certain peanut allergens cross-react in the body with other, non-peanut allergens, skilled practitioners will appreciate that the presently described compositions may be useful for treating other types of allergies. For example, the compositions and methods described herein that include peanut allergen(s) may be useful for treating allergies to birch tree pollens. Alternatively or in addition, the compositions and methods may be useful for treating allergies to pitted fruits, such as peaches and peach-related fruits and products.

The invention will be described with reference to the following examples that are intended to be illustrative only and not limiting.

### Examples

### Example 1

### GLA and Peanut Allergen Co-Administered Sublingually Provides a Protective Effect Against Peanut Allergy

The present Example describes in vivo mouse studies demonstrating that a combination of GLA and peanut allergen(s), when administered sublingually, confers protection against subsequent challenge with peanut allergens. All experiments were performed in mouse model C3H/HeOuJ. Briefly, mice were sensitized with 1 mg crude peanut extract (CPE), and/or 10 μg cholera toxin (CT) on days 0, 1, 2, 7, 14, and 21. Mice were then treated with CPE (0.5 μg, 5.0 μg, or 50 μg), GLA (aqueous formulation; GLA-AF), and methyl cellulose (MC) (1.875%) sublingually on days 28, 35, 42, and 49. Finally, the mice were challenged with 500 μg CPE intraperitoneally (IP) on day 56. The experiment was terminated on day 57. The experimental protocol is summarized in the below Table 1.

TABLE 1
Mouse allergen challenge experiment

| | | | | | | Challenge | |
|---|---|---|---|---|---|---|---|
| | Sensitization | | | Treatment | Challenge | Dose and | Termination |
| Group | (PO) | N | Treatment | Days | Day | Route (IP) | Day |
| A | Saline + | 8 | 1.875% MC | 28, 35, 42, | 56 | 500 μg | 57 |
| | CT (10 μg) | | | and 49 | | CPE | |
| B | CPE | 11 | 1.875% MC | | | | |
| C | (1.0 mg) + | 10 | GLA-AF | | | | |
| | CT (10 μg) | | (2.0 μg) + | | | | |
| | | | 1.875% MC | | | | |
| D | | 10 | GLA-AF | | | | |
| | | | (2.0 μg) + | | | | |
| | | | 1.875% MC | | | | |
| | | | with 0.5 μg CPE | | | | |
| E | | 10 | 1.875% MC | | | | |
| | | | with 0.5 μg CPE | | | | |
| F | | 10 | GLA-AF | | | | |
| | | | (2.0 μg) + | | | | |
| | | | 1.875% MC | | | | |
| | | | with 5 μg CPE | | | | |
| G | | 10 | 1.875% MC | | | | |
| | | | with 5 μg CPE | | | | |
| H | | 10 | GLA-AF | | | | |
| | | | (2.0 μg) + | | | | |
| | | | 1.875% MC | | | | |
| | | | with 50 μg CPE | | | | |
| I | | 10 | 1.875% MC | | | | |
| | | | with 50 μg CPE | | | | |

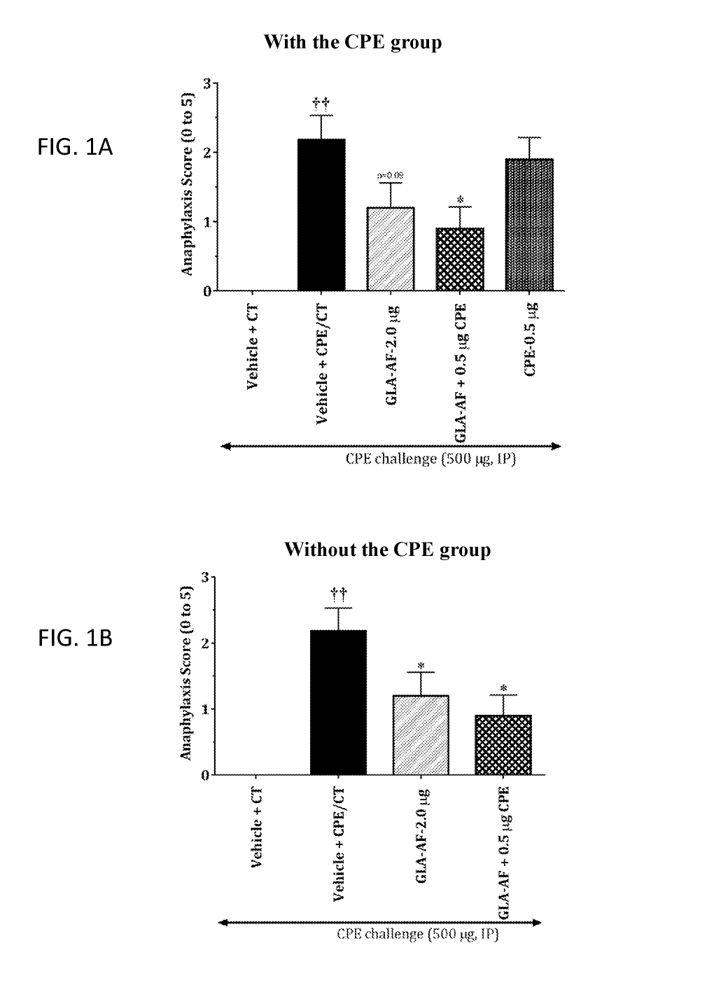

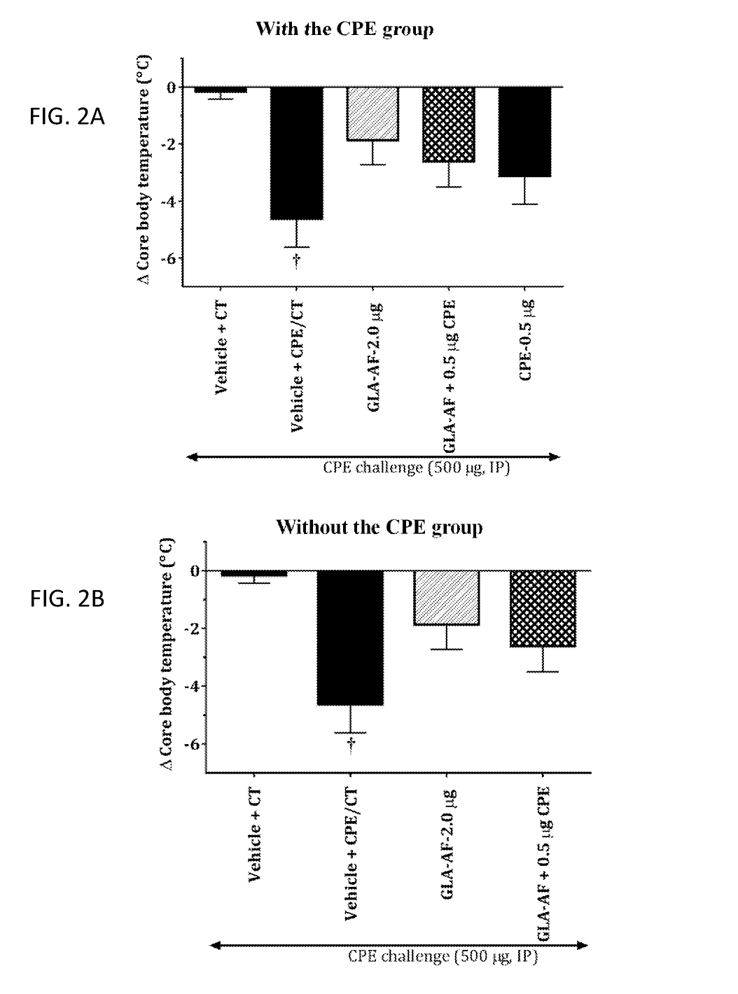

Results are provided in FIGS. 1A, 1B, 2A, and 2B. In each figure, the x-axes provide a description of the treatments administered to a given group of mice. All mice were challenged with 500 μg CPE intraperitoneally, which is indicated below the x-axes. In FIGS. 1A and 1B, the y-axes provide the anaphylaxis score observed in the mice on a scale of 0 to 5 for each treatment. In FIGS. 2A and 2B, the y-axes provide the change in core body temperature observed in the mice following challenge. A severe reaction is manifested as an increase in anaphylaxis score (FIGS. 1A and 1B) or a drop in body temperature (FIGS. 2A and 2B). The greater the decrease in core temperature, or increase in anaphylaxis, then the more severe the reaction. A protective affect is noted when, following IP challenge, a less severe drop in body temperature or a lower anaphylaxis score is observed.

Methods: For sublingual administration, a needleless syringe was used to apply 5 μl of compound sublingually under the tongue of anesthetized mice. The mice were monitored for 10 minutes to allow time for the dose to be absorbed, before the mice were returned to their cages.

Saline was 0.9% NaCl. CPE was formulated in DPBS (Dulbecco's Phosphate Buffered Saline), and diluted in saline. GLA-AF was formulated by mixing GLA and DPPC in a 1:2 molar ratio in water. Immediately before dosing, methylcellulose was added to the final concentration to create a gelatinous mixture. The mixture was vortexed vigorously and then the mixture was sonicated for 10 minutes.

For co-formulated CPE and GLA-AF, CPE was diluted to 0.4 mg/ml in saline. A 1:2 molar ratio mixture GLA-AF:DPPC in water was added to the 0.4 mg/mL CPE mixture to yield a 0.8 mg/mL GLA/0.2 mg/mL CPE solution. Methylcellulose was then added to the 0.8 mg/mL GLA/0.2 mg/mL CPE solution to make a final solution of 0.4 mg/mL GLA/0.1 mg/mL CPE/1.875% methylcellulose solution. This mixture was vortexed vigorously and then sonicated for 10 minutes. Mice were administered 5 μL of this final solution sublingually, which delivered a dose of 2.0 μg GLA-AF+0.5 μg CPE.

### Example 2

### Sublingual Co-Administration of GLA and Antigen Induces Antigen-Specific T Cell Proliferation in the Draining Cervical Lymph Nodes

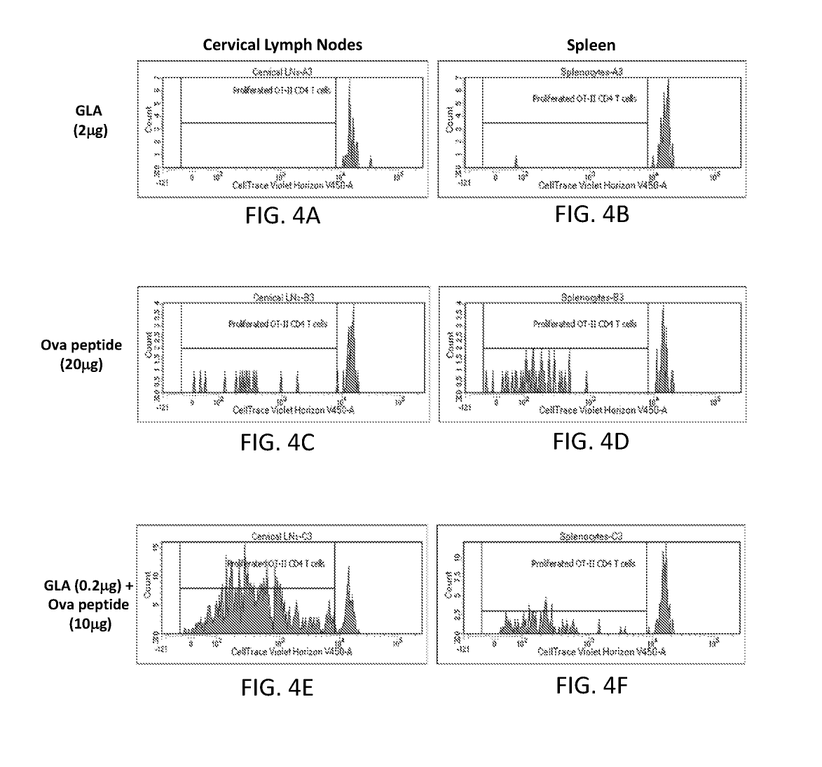

Ova-specific OT-II TcR transgenic T cells were labeled with cell-tracker violet dye and adoptively transferred into syngeneic wild type mice. Mice then received sublingual administration of GLA (0.2 μg) alone (FIGS. 4A and 4B), OVa₃₂₃₋₃₃₉ peptide (10 μg) alone (FIGS. 4C and 4D), or GLA co-administered with Ova peptide (FIGS. 4E and 4F). Four days later, the draining cervical lymph nodes (FIGS. 4A, 4C, and 4E) and spleen (FIGS. 4B, 4D, and 4F) were removed and OT-II T cell proliferation (via the decrease in cell-tracker dye fluorescence) was measured by flow cytometry. As shown in FIGS. 4A-4F, sublingual administration of GLA with Ova antigen, but not GLA or antigen alone, induces antigen-specific T cell proliferation in the draining lymph nodes but not the spleen.

Methods. Sublingual administration was performed as described above in Example 1.

Formulations of Ova peptide and/or GLA included methylcellulose as described in Example 1. Mixtures of methylcellulose+Ova peptide, and methylcellulose+Ova peptide+GLA were prepared the day before dosing.

### Example 3

### Antigen-Specific T Cell Proliferation in the Draining Cervical Lymph Nodes after Sublingual Co-Administration of GLA and OVa₃₂₃₋₃₂₉ Peptide

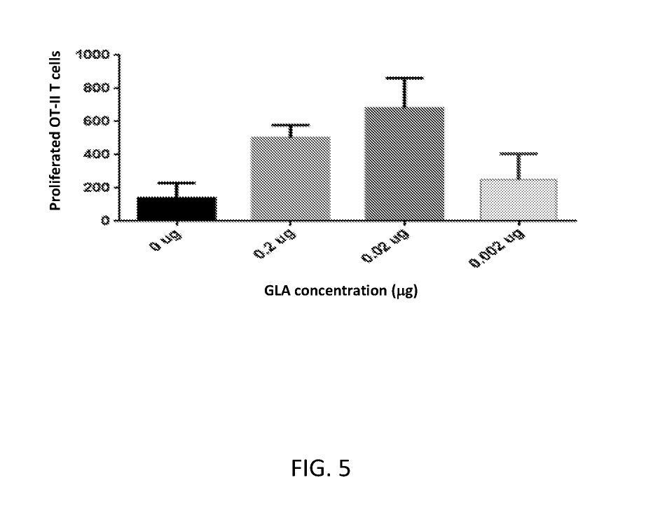

Ova-specific OT-II TcR transgenic T cells were labeled with cell-tracker violet dye and adoptively transferred into syngeneic wild type mice. Mice then received sublingual administration of GLA (0.2 μg, 0.02 μg or 0.002 μg) co-administered with OVa₃₂₃₋₃₃₉ peptide or peptide without GLA (0 mg). Four days later, cervical lymph nodes were removed and OT-II T cell proliferation (via the decrease in cell-tracker dye fluorescence) was measured by flow cytometry. As shown in FIG. 5, sublingual administration of GLA with antigen enhances antigen-specific T cell proliferation in the draining lymph nodes and in a dose-dependent manner.

Methods. Sublingual administration was performed as described above in Example 1.

Formulations of Ova peptide and/or GLA included methylcellulose as described in Example 1. Mixtures of methylcellulose+Ova peptide, and methylcellulose+Ova peptide+GLA were prepared the day before dosing.

### Example 4

### IL-6 Expression is Increased at the Site of Sublingual Administration

mRNA gene expression analysis of sublingual mucosal brushings from Non-Human Primates (NHPs) treated with 3 different doses of GLA revealed a dose-dependent expression of IL-6 six hours after sublingual administration.

GLA was formulated in DPPC (1,2-dipalmitoyl-sn-glycero-3-phosphocholine) as GLA:DPPC 1:2 (M:M) in Millipore (sterile) water. Animals were sedated with ketamine (5 mg/kg to 10 mg/kg)/dexmedetomidine (0.01 mg/kg to 0.016 mg/kg), IM (intramuscularly), while in their cage. Once sedated, they were brought into the laboratory and placed onto a circulating water heating pad (Gaymar). Sublingual (right side) and left cheek pouch mucosal brushings were taken 15 minutes prior to dosing using Copan FLOQSwabs™ and placed into RLT buffer (QIAmp Minikit Plus, Qiagen), vortexed and immediately frozen on dry ice. Sublingual dosing of GLA (100 μl dosed via syringe) was performed under the right side of the animals tongue and allowed to absorb into the mucosa for three minutes. Further sublingual and cheek brushings were obtained at 6 hr, 24 hr and 48 hr post dosing.

RNA processing was performed from the cheek and sublingual swabs using a QIAmp Minikit Plus (Qiagen) which includes a gDNA (genomic DNA) eliminator column. Gene expression was quantified using the Comparative CT Method, which calculates the relative gene expression quantitation between a target gene and an endogenous control. All target genes were quantitated relative to the expression of the housekeeping gene ARL1. Using the relative expression, the fold of baseline expression was calculated as [(Relative gene expression at time point of interest)/(Baseline relative gene expression)].

Expression was localized to the sublingual mucosa as IL-6 was not detected in brushings from the cheek or in the blood at this time point. IL-6 expression was not detected 24 hours after sublingual administration of GLA. Together the data suggest that the current formulation of GLA acts very locally within the oral cavity, does not last longer than 1 day and has a potential window of activity between 0.1 and 5m. The results also suggest that IL-6 expression may be a biomarker for GLA activity.

### Example 5

### Basophil Activation Test

The present specification provides a basophil activation test. There are at least two uses for the test. The first relates to its use in the clinic, e.g., a one tube assay for measuring biomarkers in the sera of peanut allergic patients. The second relates to its use as an assay to measure the potency (allergenicity) of a peanut extract, and which can be used to standardize the potency of different batches of peanut extract that will be used in generating compositions described herein.

Generally, an exemplary test can be performed as follows. Whole blood sample from peanut allergic patients is obtained, and basophils are primed by diluting the blood 1:1 in a BAT buffer (Basophil Activation Test Buffer), which contains 4 ng/mL rhIL-3 and 5000 IU/mL heparin (to a final concentration of 2 ng/mL rhIL-3), and incubating the whole blood cells at 37° C. for 10 minutes. The activated basophils express IgE antibody. In an antigen stimulation step, 600 μl of the primed whole blood is added to 600 μl of a 2× antigens preparation (e.g., peanut antigen preparation) in BAT buffer, and the mixture is incubated for 30 minutes in a 37° C. water bath. The addition of the antigen stimulates (and degranulates) the cells. The basophils are then surface stained, lysed and fixed. The basophils are then analyzed by fluorescence-activated cell sorting (FACS), and visualization can be accomplished by, e.g., CD63: lysosomal associated glycoprotein. The following antibody panel represents an exemplary basophil antibody cocktail that can be used for monitoring basophil activation:

| | |
|---|---|
| i. FITC | IgE |
| ii. PE | CD203c |
| iii. PE-Cy7 | CD123 |
| iv. APC | Lin* |
| v. V450 | CD63 |

Lin* = Human Hematopoietic Lineage cocktail which contains CD2, CD3, CD14, CD16, CD19, CD56, CD235a

Up-regulation of basophil proteins, or an increased rate of protein up-regulation, or overexpression of one or more of these markers over a longer period of time may be indicative of a stronger allergic response, or a more potent (more allergenic) antigen preparation.

When the sensitized basophils are exposed to antigen (e.g., peanut allergen), IgE levels are expected to decrease over time, as the basophil becomes desensitized.

Basophils are typically identified by specific markers such as CCR3⁺/CD3⁻, CD123⁺/HLA-DR⁻, IgE⁺/CD203c⁺, CD63⁺. Both CD63 and CD203c are commonly used markers for in vitro basophil activation measurement. In some experiments, the expression level of CD203c peaked earlier than CD63 in activated basophils, and thus the timing of detection may be important. Further, IL3 priming enhances CD63 expression. The activation of CD203c is transient and more rapid than the expression of CD63, so assays that use CD203c require careful consideration of the timing for detection. Accordingly, a CD203c-based basophil activation test may best be performed within 4 h after taking the blood samples. The robust assay based on IgE-dependent upregulation of a cell surface antigen on basophils was the CD63-test, and CD63 may be more specific for allergic (IgE-dependent) reactions and may be less susceptible to non-specific upregulation by cytokines or other factors. One way to measure basophil activation would be to employ both CD63 and CD203c.

In the first use of the test, i.e., in the clinic, the whole blood sample comes from a patient in the clinic, and the antigen stimulation step involves adding a 2× antigen preparation made from peanut extract or a known cocktail of peanut peptides. Then a basophil antibody cocktail is added to look for biomarker expression. An exemplary antibody cocktail includes antibodies that bind one of more of the following biomarkers: IgE, CD203c, HLS-DR, CD123, CD63, and Lin. A patient with a more severe peanut allergy will produce a blood sample that causes a result in the basophil activation test where the activated basophils express higher levels of the basophil markers, including one or more of IgE, CD203c, HLS-DR, CD123, CD63, and Lin; express higher levels of one or more of the markers for a longer period of time; or express levels of one or more of the markers that peak sooner.

In the second use of the test, e.g., to measure potency of a peanut extract, sera from peanut allergic donors can be spiked with a known quantity of a known anti-antigen antibody (e.g., anti-Ara h2 antibodies) to provide a standard, then the “antigen stimulation” step will entail adding the synthesized peanut extract to be tested. Then the “basophil antibody cocktail” will be added to look for biomarker expression (which should match an expected standard). Exemplary biomarkers include IgE, CD203c, HLS-DR, CD123, CD63, Lin. A more potent (i.e., more allergenic) peanut extract will produce a result in the basophil activation test where the activated basophils express higher levels of basophil activation markers, including one or more of IgE, CD203c, HLS-DR, CD123, CD63, and Lin; express higher levels of one or more of the markers for a longer period of time; or express levels of one or more of the markers that peak sooner.

1. A pharmaceutical composition, comprising a plurality of glucopyranosyl lipid adjuvant (GLA) particles, 1,2-dipalmitoyl-sn-glycero-3-phosphocholine (DPPC), and a therapeutically effective amount of peanut proteins, in an aqueous carrier, wherein the molar ratio of GLA to DPPC is in a range of about 1:1 to about 1:3 and at least some of the peanut proteins are disposed in at least one lipid particle or free in the aqueous carrier, or both.

2. A pharmaceutical composition, comprising glucopyranosyl lipid adjuvant (GLA) and peanut proteins, wherein the pharmaceutical composition is in a solid or semi-solid dosage form.

3. The pharmaceutical composition of claim 1, wherein the lipid particle is a liposome or a micelle.

4. (canceled)

5. The pharmaceutical composition of claim 1, wherein the GLA particles in the plurality have a Z-Average diameter of about 16 nm to about 1800 nm.

6. (canceled)

7. The pharmaceutical composition of claim 1, wherein the concentration of GLA in the composition is from about 0.01 mg/mL to about 5 mg/mL.

8-10. (canceled)

11. The pharmaceutical composition of claim 1, wherein the concentration of peanut proteins in the composition is about 5 μg/mL to about 25,600 μg/mL.

12-13. (canceled)

14. The pharmaceutical composition of claim 1, wherein the peanut proteins comprise one or more of peanut allergen components Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17.

15-19. (canceled)

20. The pharmaceutical composition of claim 2, wherein the pharmaceutical composition is a semi-solid.

21. The pharmaceutical composition of claim 2, wherein the pharmaceutical composition is in the form of a tablet, a gel capsule, a thin film, or is a multi-particulate granule or coated microsphere packaged in a sachet form.

22. (canceled)

23. The pharmaceutical composition of claim 1, wherein the pharmaceutical composition is formulated as a sublingual drop or spray.

24-38. (canceled)

39. A method of treating an allergy in a patient, comprising administering sublingually to a patient having an allergy a plurality of glucopyranosyl lipid adjuvant (GLA) particles and peanut proteins, in amounts effective to treat the allergy in the patient.

40. The method of claim 39, further comprising administering sublingually to the patient 1,2-dipalmitoyl-sn-glycero-3-phosphocholine (DPPC).

41. The method of claim 39, wherein the plurality of GLA particles is formulated as a first pharmaceutical composition and the peanut proteins are formulated as a second, separate pharmaceutical composition.

42-44. (canceled)

45. The method of claim 39, wherein the GLA and the peanut proteins are formulated in a single pharmaceutical composition comprising a plurality of GLA particles and peanut proteins in an aqueous carrier.

46. The method of claim 45, wherein the pharmaceutical composition further comprises 1,2-dipalmitoyl-sn-glycero-3-phosphocholine (DPPC).

47-50. (canceled)

51. A method of treating an allergy in a patient, comprising administering sublingually to a patient having an allergy a pharmaceutical composition of claim 2.

52-60. (canceled)

61. A method of making a pharmaceutical composition, the method comprising:
a. co-dissolving glucopyranosyl lipid adjuvant (GLA) and 1,2-dipalmitoyl-sn-glycero-3-phosphocholine (DPPC) at a 1:2 molar ratio in chloroform to thereby form a GLA/DPPC mixture;
b. adding peanut allergen to the GLA/DPPC mixture, to thereby form a GLA/DPPC/peanut proteins mixture;
c. removing the chloroform from the GLA/DPPC/peanut proteins mixture;
d. adding water to the GLA/DPPC/peanut proteins mixture; and
e. agitating the GLA/DPPC/peanut proteins mixture, to thereby form a pharmaceutical composition.

62. A method of making a pharmaceutical composition, the method comprising:
a. co-dissolving glucopyranosyl lipid adjuvant (GLA) and 1,2-dipalmitoyl-sn-glycero-3-phosphocholine (DPPC) at a 1:2 molar ratio in chloroform to thereby form a GLA/DPPC mixture;
b. removing the chloroform from the GLA/DPPC mixture;
c. adding water to the GLA/DPPC mixture;
d. agitating the GLA/DPPC mixture; and
e. adding peanut proteins to the GLA/DPPC mixture, to thereby form a pharmaceutical composition.

63. A method of making a pharmaceutical composition, the method comprising:
a. mixing glucopyranosyl lipid adjuvant (GLA) and 1,2-dipalmitoyl-sn-glycero-3-phosphocholine (DPPC) at a 1:2 molar ratio in water to thereby form a GLA/DPPC mixture;
b. agitating the GLA/DPPC mixture at 70° C.; and
c. adding peanut proteins to the GLA/DPPC mixture, to thereby form a GLA/DPPC/peanut proteins mixture;
to thereby form a pharmaceutical composition.

64. A method of making a pharmaceutical composition, the method comprising:
a. mixing glucopyranosyl lipid adjuvant (GLA) and a surfactant;
b. adding water to the GLA/surfactant mixture;
c. agitating the GLA/surfactant mixture; and
d. adding peanut proteins to the GLA/surfactant mixture, to thereby form a pharmaceutical composition.

65.-78. (canceled)
